(19) United States
(12) Patent Application Publication
(10) Pub. No.: US 2002/0072071 A1
(43) Pub. Date: Jun. 13, 2002

(54) METHOD FOR THE DETECTION OF PSA-ACT COMPLEXES

(76) Inventors: ROSEMARIE KIENTSCH-ENGEL, FELDAFING (DE); ELLEN MOSSNER, TUTZING (DE); OLLE NILSSON, GOTEBORG (SE)

Correspondence Address: MARILYN L. AMICK, ROCHE DIAGNOSTICS CORPORATION, 9115 HAGUE ROAD, P.O. BOX 50457, INDIANAPOLIS, IN, 46250, US

(21) Appl. No.: 09/192,430

(22) Filed: Nov. 16, 1998

(30) Foreign Application Priority Data
Nov. 17, 1997 (DE) 197 50 916.9

(51) Int. Cl. G01N 33/53; G01N 33/574; G01N 33/537; G01N 33/543; G01N 33/48

(52) U.S. Cl. 435/7.1; 435/7.23; 435/7.93; 435/7.95; 436/63; 436/64

(57) ABSTRACT

For the immunological determination of PSA-ACT complexes in a sample to be examined in which the sample is contacted with at least one antibody that specifically binds the complexes or their components or with specifically binding fragments thereof in which the antibody is bound to a solid phase or can be bound to a solid phase, a test liquid is used according to the invention which contains 0.5 to 2.5 mol/l of an alkali chloride at least during contact between the sample and the solid phase. A buffer system according to the invention for a PSA-ACT test contains 0.5 to 2.5 mol/l, preferably 1.5 to 2.0 mol/l alkali chloride in addition to other common buffer substances. A test kit according to the invention for the detection of PSA-ACT complexes in the serum of patients contains

a) a PSA-specific antibody and

b) an ACT-specific antibody wherein one of the antibodies a) or b) can be bound to a solid phase or is bound to a solid phase and the other antibody a) or b) has a label via which the detection reaction takes place, and at least one buffer system as claimed in one of the claims 10 to 15 as the buffer.

### DESCRIPTION

The present invention concerns a method for the immunological determination of PSA-ACT complexes, buffer systems for such tests and a test kit for the detection of PSA-ACT complexes in the serum of patients.

The prostate-specific antigen, PSA is formed by prostate epithelial cells and has the enzymatic activity of a neutral serine protease. PSA is a component of seminal fluid but small amounts also pass into the human blood circulation. The enzymatically active PSA is inactivated in serum by various serine protease inhibitors by the formation of covalent complexes. Most of the immunologically detectable PSA is bound in the serum to α1-antichymotrypsin (60 to 95%).

α1-Antichymotrypsin (ACT) is a glycoprotein which plays an important role in the control of inflammations. ACT occurs in human serum in a 10,000-fold higher concentration than PSA.

The complexed PSA-ACT is present as the main form of the immunologically detectable total PSA in the serum in addition to free PSA. It was found that, in comparison to patients with benign prostate hyperplasia, increased levels of complexed PSA mainly occur in patients with malignant prostate tumours (Japanese unexamined laid-open patent application 62-46263).

The detection of PSA-ACT therefore serves among others to screen for and detect malignant prostate carcinomas as well as to monitor therapeutic measures. In this connection the amount of PSA-ACT that is present correlates above a threshold value very strongly with the presence or malignancy and size of prostate carcinomas.

PSA-ACT complexes have therefore already been detected by means of sandwich assays using specific antibodies. For this firstly an antibody coupled to a solid phase is used which is specific for one of the two complex components in addition to a labelled antibody which is specific for the other of the two complex components, the detection finally occurring via the label of the second antibody (e.g. WO92/01936, JP62-46263, Pettersson et al., Clin. Chem. 41/10 (1995), 1480-1488, Leinonen et al., Clin. Chem. 39/10 (1993), 2098-2103).

Unfortunately it has now turned out that substances are present in the serum which can severely interfere with this test. This is mainly cathepsin G which, like PSA, is a serine protease and is also bound by the inhibitor α1-antichymotrypsin (ACT) and thereby inactivated. However, cathepsin G has the additional unpleasant property of being very sticky and binds for example to the solid phase in immunological tests. Hence, in the presence of cathepsin G which is formed in large amounts especially when inflammations are present, complexes between the solid phase, cathepsin-G-ACT and the labelled anti-ACT antibody are formed in the current tests which simulate the presence of PSA-ACT complexes and falsely increased values are measured (see e.g. Leinonen, 1993, supra which reports tests in which PSA-ACT/PSAₜₒₜₐₗ of larger than 1 was measured and even false positive values were found in the sera of women).

The object of the present invention was therefore to increase the specificity of the detection of PSA-ACT complexes and to avoid interference especially by cathepsin G.

This object is achieved by a method for the immunological determination of PSA-ACT complexes in a sample to be examined in which the sample is contacted with an antibody that specifically binds the complexes or their components or with specifically binding fragments thereof, the antibody being bound to a solid phase or capable of being bound to a solid phase characterized in that at least during contact between the sample and the solid phase a test liquid is used which contains 0.5 to 2.5 mol/l of an alkali chloride.

The sample which is to be examined for the presence of PSA-ACT complexes is a body fluid in which increased PSA-ACT complex concentrations are found when malignant prostate tumours are present.

Within the scope of the invention it was found that surprisingly a relatively high salt content in the test buffer used substantially prevents unspecific bindings and does not interfere with the immunological reaction of the antibodies used. Values are obtained with the aid of the method according to the invention which give a very high correlation of the value calculated for the difference of total PSA and free PSA with the actually measured value which argues for the quality of the new method.

Within the scope of the present invention a buffer is preferably used which contains 1.5 to 2.0 mol/l alkali chloride. Sodium or potassium chloride are particularly preferably used as alkali chlorides.

Furthermore it was found that addition of an alkali tartrate to the experimental buffer is of additional advantage. 0.01 to 0.05 mol/l alkali tartrate is preferably used according to the invention. Sodium tartrate is a particularly preferred tartrate. This is used especially in amounts of 0.02 to 0.03 mol/l in the buffer.

Another method of further reducing false-positive results is to use a buffer which additionally contains modified thermo-BSA (thermo bovine serum albumin) (e.g. WO 95/17668) and preferably in an amount of 0.2 to 0.5 g/l.

Suitable solid phases for immunoassays are known to a person skilled in the art. The most common solid phases are mentioned here merely as an example such as plastic tubes, polystyrene beads, latex particles, microtitre plates, glass beads etc.

Means of binding antibodies to such solid phases are also known to a person skilled in the art. Reference is made here to the binding pairs biotin/(strept)avidin, hapten/antibody, lectin/carbohydrate as examples where in each case one partner is coupled to the solid phase and the other partner is present associated with the antibody.

These binding pairs are, however, only to be understood as examples, other binding pairs can be used likewise within the scope of the invention such as a direct e.g. adsorptive binding of the capture antibody to the solid phase.

After coupling of the complexes via the antibody to the solid phase, the bound complexes can be detected in any manner known to a person skilled in the art. In a preferred embodiment an immunoassay is used in which the steps

a) coupling an antibody specific for (1) PSA or for (2) ACT to a solid phase,

b) adding a sample to be examined to the solid phase and

c) detecting the presence of PSA-ACT complexes by adding a labelled antibody which in the case of a)(1) is specific for ACT and in the case of a)(2) is specific for PSA and detecting by means of this antibody label

are carried out in a suitable order in which, according to the invention, at least in step b) a buffer is used with the aid of which the test liquid is adjusted to a content of 0.5 to 2.5 mol/l of an alkali chloride.

Thus the preferred method according to the invention comprises a sandwich immunoassay like those which in principle have previously been used. All suitable test formats for sandwich immunoassays including known solid phases, systems for coupling capture antibodies to solid phases, detection by means of detection antibody labels can be used within the scope of the invention provided that at least when the sample to be examined is contacted with the solid phase, with the solid phase-bound antibody or with the antibody which is to be bound to the solid phase, the stated salt content is present. Also the order in which steps a) and b) are carried out is not critical in the preferred method according to the invention.

Also the choice of label associated with the detection antibody in step c) is uncritical within the scope of the invention. All labels which are generally suitable for sandwich immunoassays can also be used in the present method. In this connection direct or indirect labels can be used whereby indirect labels are understood as those which are in turn detected via a further substance such as a hapten which is detected by a further antibody (e.g. DIG-Anti-DIG).

Examples of direct labels are enzyme labels, labels capable of electrochemiluminescence such as Ru chelates, gold labels and fluorophores.

In a further preferred embodiment of the invention steps a) and b) of the method according to the invention are carried out simultaneously and namely in a buffer which contains the necessary salt component and optionally tartrate or/and thermo-BSA. This procedure simplifies and accelerates the detection according to the invention.

It is obvious within the scope of the invention that antibodies are used whose specificity and bindability are not impaired by the salt concentration of the buffer. It is easy to establish by simple experiments whether an antibody under consideration fulfils this condition. Moreover it is advantageous to use monoclonal antibodies in the method according to the invention. Finally antibodies can also be used within the present invention which specifically bind PSA or ACT in the complexed PSA-ACT form. Such antibodies are included under the inventive term “antibodies specific for PSA or ACT” and are described for example in the German Patent Application 196 41 560.8 to which reference is herewith made. It is obvious for persons skilled in the art that also in this case a sandwich assay can only be carried out when both antibodies used recognize different epitopes of the PSA-ACT complex or only one complex-binding antibody and one antibody specific for one of the two parts of the complex is used. In the case of all the antibody types described above it is also additionally possible to use suitable fragments for immunoassays (Fab, Fab′, F(ab)₂′) and also chimeric antibodies.

All details concerning the test format are only intended as an illustration and are not to be interpreted as a definitive description. As already setforth above all immunoassays and in particular sandwich assays are suitable for the present invention provided the inventive concentration of alkali chloride is used (at least in step b)) but optionally also in other steps of the assay.

However, within the framework of the invention it is also possible to carry out a test in which for example an antibody that is only specific for the complex PSA-ACT is used as a capture antibody and labelled PSA-ACT complexes are added along the lines of a competitive assay. However, all other suitable test formats in which false-positive results for PSA-ACT complexes are obtained due to unspecific binding of substances contained in the sample to the solid phase, can also be positively influenced by the inventive addition of salt(s) or/and other substances to the test liquid and are therefore suitable methods within the framework of the present invention.

A further subject matter of the present invention is a buffer system for a PSA-ACT test containing 0.5 to 2.5 mol/l and in particular 1.5 to 2.0 mol/l alkali chloride in addition to other common buffer substances. In the buffer system according to the invention it is preferred that it contains sodium chloride or/and potassium chloride as the alkali chloride which have proven to be particularly suitable. In addition the buffer system according to the invention preferably contains an alkali tartrate, especially in amounts of 0.01 to 0.05 mol/l and preferably 0.02 to 0.03 mol/l. The alkali tartrate that is particularly preferred according to the invention is sodium tartrate. The inventive buffer system additionally advantageously contains modified thermo-BSA especially in amounts of 0.2 to 0.5 g/l.

The buffer system of the invention can be used to carry out PSA-ACT tests, which are preferably designed as a sandwich immunoassay but are not limited thereto, almost without interference by other substances contained in the blood, serum or other body fluids. The salt content of the buffer system as well as the presence of modified thermo-BSA prevents substances that bind unspecifically, such as the “sticky” cathepsin G, from unspecifically bridging the antibody or unspecifically binding the labelled antibody or another detection substance to the solid phase.

Therefore a further subject matter of the invention is the use of the buffer system according to the invention for the specific immunological determination of PSA-ACT complexes in a sample to be examined.

The sample to be examined is a body fluid which has an increased concentration of the PSA-ACT complex when a malignant prostate tumour is present in particular whole blood, plasma or serum, serum being preferably examined. A further subject matter of the invention is a test kit for the detection of PSA-ACT complexes in the serum of patients. The test kit preferred according to the invention contains suitable substances for PSA-ACT detection as a sandwich assay i.e.

a) an antibody specific for PSA and

b) an antibody specific for ACT in which one of the antibodies can be bound or is bound to a solid phase and the other antibody has a label by which the detection reaction is carried out. The test kit according to the invention additionally contains an inventive buffer system as the buffer containing alkali chloride in particular sodium or potassium chloride and optionally alkali tartrate preferably sodium tartrate and optionally modified thermo-BSA. The amounts of these substances present in the buffer system as well as the amounts that are preferably used have already been stated in the above description.

The test kit according to the invention preferably also contains a solid phase to which one of the two antibodies, the capture antibody, is bound or can be bound e.g. by means of a binding pair as already described above for the method according to the invention.

The type of solid phase is not limiting, all solid phases suitable for sandwich assays can be present in the test kit.

The explanations and examples already given above for the method according to the invention also apply to the antibody and the antibody label (detection antibody) that are used.

The method according to the invention, the buffer system as well as the test kit enable a very reliable and interference-free immunological detection of PSA-ACT complexes. Thus in the case of the sandwich test according to the invention it was found that the sum of the measured PSA-ACT complex and of free PSA (determined by known methods) corresponded to the total PSA. Also in women correct PSA-ACT values of almost zero are measured with the aid of the method according to the invention even when inflammations were present which greatly increase the amount of cathepsin G, ACT and the complex ACT-cathepsin G. The amounts of salt used do not interfere with the test so that the buffer systems according to the invention only have a positive effect.

The following examples are intended to further elucidate the invention.

### EXAMPLE 1

Effect of Sodium Chloride on Test Interference by Cathepsin G

The reduction of interference in the PSA-ACT test was examined as follows:

Streptavidin coated polystyrene tubes (Boehringer Mannheim GmbH, order No. 1 144 553) served as the solid phase. The anti-PSA monoclonal antibody clone M10 was added as a biotinylated Fab-fragment at a concentration of 1.5 μg/ml to the incubation buffer of the Enzymun-test® PSA (Boehringer Mannheim GmbH, order No. 1 555 332) together with increasing amounts of NaCl (=buffer 1). 0.025 to 0.2 mol/l Na tartrate were additionally added to the variant containing 2 mol/l NaCl (=buffer 2). The test was carried out on an ES 300 automated analyser (Boehringer Mannheim GmbH) with incubation periods of 45, 30 and 30 min for the sample, conjugate and substrate reaction respectively in volumes of 700 μl in each case; washing was carried out at intensity 8 between the individual incubation steps (Enzymun-test® wash solution, Boehringer Mannheim GmbH, order No. 1 059 475).

The PSA-ACT standard material purchased from the Scripps Co. (cat. No. P 0624.CH. 516664) was used at concentrations of 0, 0.61, 2.44, 13.6 and 54.67 ng/ml. Anti-ACT clone 53 served as the monoclonal detection antibody after conjugation of the IgG to horseradish peroxidase. The colour reaction was carried out in the usual manner with 1.9 mmol/l ABTS® (2,2′-azino-di-[3-ethyl-benzthiazoline sulfonic acid(6)]diammonium salt) and 3.2 mmol/l sodium perborate in 100 mmol/l phosphate/citrate buffer pH 4.4.

0.5 to 5 mg cathepsin G/l was added to pooled serum of healthy male or female blood donors and the influence of the various buffer systems was examined. It turned out (see table 1) that the addition of 2 mol/l NaCl greatly reduces test interference (buffer 1) and the further addition of small amounts of sodium tartrate further reduces the interference (buffer 2).

TABLE 1

| | | | | | | buffer 2 + | | |
|---|---|---|---|---|---|---|---|---|
| | buffer 1 + | | | | | di-sodium tartrate | | |
| | NaCl [mol/l] | | | | | [mol/l] | | |
| Sample | 0.15 | 0.5 | 1.0 | 1.5 | 2.0 | 0.025 | 0.1 | 0.2 |
| male serum* + | | | | | | | | |
| cathepsin G | | | | | | | | |
| [mg/l] | | | | | | | | |
| 0 | 0.105 | 0.101 | 0.095 | 0.094 | 0.087 | 0.087 | 0.096 | 0.100 |
| 0.5 | 0.114 | 0.106 | 0.101 | 0.099 | 0.086 | 0.087 | 0.098 | 0.102 |
| 1 | 0.130 | 0.114 | 0.113 | 0.105 | 0.091 | 0.089 | 0.102 | 0.110 |
| 2 | 0.152 | 0.122 | 0.119 | 0.106 | 0.095 | 0.091 | 0.110 | 0.113 |
| 5 | 0.196 | 0.145 | 0.139 | 0.118 | 0.108 | 0.094 | 0.126 | 0.126 |
| female | | | | | | | | |
| serum** + | | | | | | | | |
| cathepsin G | | | | | | | | |
| [mg/l] | | | | | | | | |
| 0 | 0.002 | 0.004 | 0.002 | 0.002 | 0.002 | 0.002 | 0.003 | 0.008 |
| 0.5 | 0.006 | 0.010 | 0.004 | 0.004 | 0.004 | 0.004 | 0.003 | 0.008 |
| 1 | 0.010 | 0.011 | 0.007 | 0.006 | 0.004 | 0.004 | 0.004 | 0.008 |
| 2 | 0.016 | 0.012 | 0.009 | 0.007 | 0.003 | 0.003 | 0.004 | 0.009 |
| 5 | 0.019 | 0.018 | 0.015 | 0.010 | 0.006 | 0.004 | 0.008 | 0.012 |

* = pooled serum of healthy male blood donors
** = pooled serum of healthy female blood donors
Data in absorbance units measured at 422 nm

### EXAMPLE 2

Influence of Other Alkali Salts on the Reduction of Interference in the PSA-ACT Test

Instead of NaCl, other alkali salts were added to the incubation buffer of the Enzymun Test® PSA and their effect on the PSA-ACT determination was measured in the presence of increasing amounts of cathepsin G. The remaining test procedure corresponds to example 1. The result is summarized in Table 2.

This experiment shows that KCl eliminates test interference almost equally as effectively as NaCl. KI and KSCN were unsuitable since they interfered with the test itself (see Table 2, lower part PSA standard). LiCl did not adequately reduce interference.

| Sample | | | | | |
|---|---|---|---|---|---|
| Serum* containing | PSA buffer with the addition of | | | | |
| cathepsin G | | KCl | KI | LiCl | KSCN |
| [mg/l] | — | 1.5 mol/l | 1.5 mol/l | 0.5 mol/l | 1 mol/l |
| 0 | 0.016 | 0.011 | 0.011 | 0.010 | 0.020 |
| 0.5 | 0.027 | 0.020 | 0.018 | 0.026 | 0.033 |
| 1.0 | 0.043 | 0.022 | 0.024 | 0.040 | 0.043 |
| 2.0 | 0.081 | 0.035 | 0.037 | 0.077 | 0.063 |
| 5.0 | 0.21 | 0.065 | 0.068 | 0.175 | 0.146 |
| PSA-ACT | | | | | |
| standard | | | | | |
| 0 ng/ml | 0.005 | 0.012 | 0.007 | 0.005 | 0.010 |
| 54.7 ng/ml | 4.128 | 4.272 | 1.680 | 4.144 | 1.411 |

*pooled serum of healthy male blood donors
data in absorbance units measured at 422 nm

### EXAMPLE 3

Addition of Modified Thermo-BSA Reduces Interference in the PSA-ACT Test

If two incubation buffers containing different concentrations of thermo-BSA are compared such as for example the incubation buffer (bottle 1) of the Enzymun-Test® NSE (Boehringer Mannheim GmbH, order No. 1660438) and PSA (Boehringer Mannheim GmbH, order No. 1555332) which contain modified thermo-BSA (described in WO95/17668) at concentrations of 0.05 and 0.35 g/l respectively and whose remaining compositions are equal, it becomes clear (see Table 3) that this component considerably contributes to the reduction of test interference.

The other test components and the procedure correspond to those of example 1.

TABLE 3

| Sample | | | |
|---|---|---|---|
| serum* containing | Incubation buffer containing thermo-BSA | | |
| cathepsin G | | | 0.035 g/l + |
| [mg/l] | 0.05 g/l | 0.35 g/l | 2 mol/l NaCl |
| 0 | 0.116 | 0.105 | 0.087 |
| 0.5 | 0.135 | 0.114 | 0.087 |
| 1.0 | 0.185 | 0.130 | 0.089 |
| 2.0 | 0.256 | 0.152 | 0.091 |
| 5.0 | 0.352 | 0.196 | 0.094 |

*pooled serum of healthy male blood donors
Data in absorbance units measured at 422 nm

### EXAMPLE 4

Serum Level of PSA-ACT in Various Groups of Patients/Test Persons

The PSA-ACT level was determined in the serum of various groups of test persons or patient groups with the test version described in example 1 using the incubation buffer to which 2 mol/l NaCl and 0.025 mol/l sodium tartrate were added. The result is shown in Table 4.

TABLE 4
PSA-ACT content of sera

| | | | | | other |
|---|---|---|---|---|---|
| | male | female | benign prostate | prostate | diseases, |
| Group | controls | controls | hyperplasia | carcinoma | male patients |
| number of | 70 | 41 | 51 | 61 | 85 |
| samples | | | | | |
| PSA-ACT | | | | | |
| [ng/ml] | | | | | |
| mean | 1.08 | 0.01 | 2.76 | 8.49 | 1.29 |
| median | 0.75 | 0.00 | 1.83 | 5.27 | 0.66 |
| minimum | 0.00 | 0.00 | 0.00 | 0.00 | 0.00 |
| value | | | | | |
| maximum | 5.26 | 0.04 | 13.03 | 69.00 | 29.10 |
| value | | | | | |

### EXAMPLE 5

Comparison of Calculated/Measured PSA-ACT

If one assumes that the total content of PSA in a sample is essentially composed of the two components free PSA and PSA-ACT, the difference of [PSA total minus PSA free] can be calculated and this result can be compared with the PSA-ACT determined in the same samples as a test for the plausibility of the PSA-ACT result. Therefore PSA total and PSA free were additionally determined in the samples used for example 4 (apart from the female controls) using the corresponding Enzymun-Tests® and the correlation of difference between these two parameters and PSA-ACT was calculated. The result is summarized in Table 5.

TABLE 5

| parameter | parameter | | number of | | | |
|---|---|---|---|---|---|---|
| 1 | 2 | group | values | r | m | b |
| PSA-ACT | tPSA-fPSA | all | 264 | 0.994 | 1.010 | 0.283 |
| (= Y value) | (= X-value) | C, male | 70 | 0.988 | 1.153 | 0.092 |
| | | BPH | 51 | 0.955 | 1.211 | 0.191 |
| | | PCa | 60 | 0.997 | 0.996 | 0.249 |
| | | other | 83 | 0.998 | 1.024 | 0.187 |
| | | diseases | | | | |

tPSA = PSA total, fPSA = PSA free
C, male = male healthy control
BPH = benign prostate hyperplasia
PCa = prostate carcinoma
other diseases = other diseases of male patients
r = correlation coefficient
m = slope of the correlation line
b = axis intercept of the correlation line

In all samples the correlation between calculated and measured PSA-ACT was almost 1. This indicates a plausible result for PSA-ACT that was obtained with the new method of determination.

1. Method for carrying out a heterogeneous immunoassay for the immunological determination of PSA-ACT complexes in a sample to be examined in which the sample is contacted with at least one antibody that specifically binds the complexes or their components or with specifically binding fragments thereof and the antibody or its fragments is bound to a solid phase or can be bound to a solid phase,
wherein
at least during contact between the sample and the solid phase, a test liquid is used which contains 0.5 to 2.5 mol/l of an alkali chloride:

2. Method as claimed in claim 1,
wherein
a buffer is used which contains 1.5 to 2.0 mol/l alkali chloride.

3. Method as claimed in claim 1 or 2,
wherein
a buffer is used which contains sodium chloride or potassium chloride as the alkali chloride.

4. Method as claimed in one of the claims 1 to 3,
wherein
alkali tartrate is additionally added to the buffer in such an amount that 0.01 to 0.05 mol/l alkali tartrate is present in the experimental mixture.

5. Method as claimed in claim 4,
wherein
sodium tartrate is used.

6. Method as claimed in claim 4,
wherein
a buffer is used which contains 0.02 to 0.03 mol/l sodium tartrate.

7. Method as claimed in one of the claims 1 to 6,
wherein
a buffer is used which additionally contains modified thermo-BSA preferably in an amount of 0.2 to 0.5 g/l.

8. Method as claimed in one of the previous claims in which the steps
a) coupling an antibody specific for (1) PSA or for (2) ACT to a solid phase,
b) adding a sample to be examined to the solid phase and
c) detecting the presence of PSA-ACT complexes by adding a labelled antibody which in the case of
a)(1) is specific for ACT and in the case of
a)(2) is specific for PSA and detecting by means of this antibody label,
are carried out
wherein
at least in step b) a buffer is present with the aid of which the test liquid is adjusted to a content of 0.5 to 2.5 mol/l of an alkali chloride.

9. Method as claimed in claim 8,
wherein
steps a) and b) are carried out simultaneously and in this process antibodies and the sample to be examined are admixed with a buffer which contains the required components.

10. Method as claimed in one of the previous claims,
wherein
monoclonal antibodies or specifically binding fragments thereof are used.

11. Buffer system for a heterologous immunological method of detection for the determination of PSA-ACT complexes in body fluids that contains 0.5 to 2.5 mol/l and preferably 1.5 to 2.0 mol/l alkali chloride in addition to other common buffer substances.

12. Buffer system as claimed in claim 11,
wherein
it contains sodium chloride or/and potassium chloride as the alkali chloride.

13. Buffer system as claimed in claim 11 or 12,
wherein
it additionally contains 0.01 to 0.05 mol/l, preferably 0.02 to 0.03 mol/l alkali tartrate.

14. Buffer system as claimed in claim 13,
wherein
it contains sodium tartrate.

15. Buffer system as claimed in one of the claims 11 to 14,
wherein
it additionally contains modified thermo-BSA.

16. Buffer system as claimed in claim 15,
wherein
it contains 0.2 to 0.5 g/l modified thermo-BSA.

17. Use of a buffer system as claimed in one of the claims 11 to 16 in a heterologous immunological detection method for PSA-ACT complexes in a sample to be examined.

18. Use as claimed in claim 17,
wherein
the detection method comprises a sandwich assay.

19. Test kit for the detection of PSA-ACT complexes in the serum of patients,
wherein it contains
a) a PSA-specific antibody and
b) an ACT-specific antibody in which one of the antibodies a) or b) can be bound to a solid phase or is bound to a solid phase, and the other antibody a) or b) has a label via which the detection reaction takes place and at least one buffer system as claimed in one of the claims 11 to 16 is present as the buffer.

20. Test kit as claimed in claim 19,
wherein
it contains a solid phase to which one of the antibodies a) or b) is bound or can be bound.
